(19) United States
(12) Patent Application Publication
(10) Pub. No.: US 2017/0056542 A1
(43) Pub. Date: Mar. 2, 2017

(54) AQUEOUS-BASED GELS, FREE-STANDING AIR FRESHENERS, AND METHODS FOR MANUFACTURING AQUEOUS-BASED GELS

(71) Applicant: The Dial Corporation, Scottsdale, AZ (US)

(72) Inventors: Alex Hamilton, Phoenix, AZ (US); Kevin Hafer, Chandler, AZ (US); Tyler Mikkelsen, Scottsdale, AZ (US)

(21) Appl. No.: 14/837,994

(22) Filed: Aug. 27, 2015

(51) Int. Cl. A61L 9/012

(52) U.S. Cl. CPC A61L 9/012

(57) ABSTRACT

Aqueous-based gels capable of free standing, free-standing air fresheners, and methods for manufacturing aqueous-based gels are provided herein. In accordance with an exemplary embodiment, an aqueous gel capable of free standing includes a C₁₂-C₂₄ fatty acid or a salt thereof, a freeze/thaw stabilizer, xanthan gum, a gluco- or galactomannan, and water. The xanthan gum and the gluco- or galactomannan are present in the aqueous-based gel in a xanthan gum:gluco- and/or galactomannan ratio in the range of about 50:50 to about 75:25.

### TECHNICAL FIELD

The present invention generally relates to gels and methods for fabricating gels, and more particularly relates to aqueous-based gels capable of free standing, free-standing air fresheners, and methods for manufacturing aqueous-based gels.

### BACKGROUND

Present day aqueous gel air fresheners often utilize caregeenan to carry a fragrance that is slowly released into the atmosphere. Carageenan is a naturally marine-sourced polymeric material. These products look like solids, although closer inspection reveals that they are rigid water gels having an outer surface that is easily penetrated during a procedure used to measure gel strength.

While carrageenan has been used widely as a gelling agent, its use poses significant challenges. Carageenan supply is typically geographically limited to South Asia. Because its overall supply can be hampered by weather, industry, and other agricultural issues, carrageenan can see wide price instability and global supply shortages. In addition, only certain grades of carrageenan can be used as gelling agents suitable for particular applications, such as air fresheners, and only certain suppliers provide these carrageenan grades, thus limiting the supply even further. In addition, grades of carrageenan can vary from year to year. Just as a year of bad weather may render grapes unsuitable for making wine, a year of bad weather may render the applicable grades of carrageenan poor for air freshener gels, further increasing production costs.

Moreover, even suitable carrageenan has drawbacks for use as a gelling agent, particularly in home care products. Carageenan is beige/brown in color and thus is difficult to dye. Dyeing of gels is important in applications such as home care products where consumers desire products that match or assimilate into their home décor. Grades of carrageenan also suffer from syneresis. Syneresis is the extraction or expulsion of free liquid (typically water) from a gel. Syneresis is problematic because the free liquid from the gel has a tendency to flow from the gel to the support upon which the gel stands, such as a countertop.

If the gel contains a dye or pigment, such as in the case of home care air fresheners, the dye or pigment tends to dissolve in the water that results from syneresis and can stain the countertop or other support upon which the gel stands. Moreover, certain grades of carrageenan cannot provide free-standing gels. Free-standing gels allow for the vaporization or other distribution of an active ingredient, such as a fragrance, from more than just one surface of the gel. Kappa carrageenan can provide a strong, free-standing gel but typically yields an undesirable amount of water as a result of syneresis. Iota carrageenan, on the other hand, does not exhibit undesirable syneresis but cannot provide a free-standing gel. A blend of both carrageenans is desirable for use in home care products, but this blend doubles the problems associated with sourcing carrageenan as explained above.

Accordingly, it is desirable to provide an aqueous-based gel capable of free standing that does not contain carrageenan. In addition, it is desirable to provide a free-standing air freshener that does not contain carrageenan. It also is desirable to provide a method for manufacturing an aqueous-based gel capable of free standing. Furthermore, other desirable features and characteristics of the present invention will become apparent from the subsequent detailed description of the invention and the appended claims, taken in conjunction with the accompanying drawings and this background of the invention.

### BRIEF SUMMARY

Aqueous-based gels capable of free standing, free-standing air fresheners, and methods for manufacturing aqueous-based gels are provided herein. In accordance with an exemplary embodiment, an aqueous gel capable of free standing includes a C₁₂-C₂₄ fatty acid or a salt thereof, a freeze/thaw stabilizer, xanthan gum, a gluco- or galactomannan, and water. The xanthan gum and the gluco- or galactomannan are present in the aqueous-based gel in a xanthan gum:gluco- or galactomannan ratio in the range of about 50:50 to about 75:25.

In accordance with another exemplary embodiment, a free-standing air freshener includes a C₁₂-C₂₄ fatty acid or a salt thereof, sodium carboxymethyl cellulose, xanthan gum, a gluco- or galactomannan, an active ingredient, and water. The xanthan gum and the gluco- or galactomannan are present in the free-standing air freshener in a xanthan gum:gluco- or galaetomannan ratio in the range of about 50:50 to about 75:25.

In accordance with a further exemplary embodiment, a method for manufacturing an aqueous-based gel capable of free standing is provided. The method includes mixing a C₁₂-C₂₄ fatty acid or a salt thereof, a freeze/thaw stabilizer, xanthan gum, and a gluco- or galactomannan with water at a temperature sufficient to form a homogeneous liquid mixture. The xanthan gum and the gluco- or galactomannan are present in a xanthan gum:gluco- or galactomannan ratio in a range of about 50:50 to about 75:25. The homogeneous liquid mixture is cooled actively or passively.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

Various embodiments of aqueous-based gels capable of free standing, free-standing air fresheners, and methods for manufacturing aqueous-based gels are provided herein. As noted, the aqueous-based gels are capable of “free standing.” In this regard, the gels are viscoelastic solids that can rest on a counter, table, or other substrate without requiring additional physical support at any of their other surfaces, such as at their side surfaces, or without requiring additional physical support from within the gel. In addition, the aqueous-based gels utilize a C₁₂-C₂₄ fatty acid or a salt thereof as a gelling agent and thus are free of carrageenan, that is, they are “carrageenan-free.” In this regard, the aqueous-based gels have no carrageenan as a gelling agent or have an amount of carrageenan as a gelling agent such that the chemical or physical characteristics of the aqueous-based gel are not visually or measurably different from an aqueous-based gel having the same composition but with no carrageenan as a gelling agent. Moreover, the aqueous-based gels contain a novel ratio of xanthan gum: gluco- or galactomannan. It unexpectedly has been found that this novel ratio of xanthan gum: gluco- or galactomannan, when used in the aqueous-based gel composition, provides an optimum gel strength not provided outside of this ratio while simultaneously providing a minimum syneresis effect.

In accordance with an exemplary embodiment, the aqueous-based gel as contemplated herein contains a C₁₂-C₂₄ free fatty acid or a salt thereof as a gelling agent. The C₁₂-C₂₄ free fatty acid or a salt thereof can be saturated or unsaturated. As used herein, a C₁₂-C₂₄ free fatty acid means a saturated or unsaturated free fatty acid, or a salt thereof, having a total number of carbon atoms in the range of from 12 to 24. In an embodiment, the free fatty acid, or the salt thereof, is a C₁₄-C₂₂ free fatty acid or a salt thereof, for example, a C₁₆-C₂₀ free fatty acid or a salt thereof. In one embodiment, the aqueous-based gel contains a mixture of C₁₆ free fatty acids or salts thereof and C₁₈ free fatty acids or salts thereof. In another embodiment, the aqueous-based gel contains C₁₆ free fatty acids or salts thereof and C₁₈ free fatty acids or salts thereof in a ratio of 45:55, respectively. It will be appreciated that the longer carbon chain free fatty acids or salts thereof may provide stronger gels depending on the remaining ingredients of the gel. Examples of free fatty acids suitable for use as a gelling agent herein include lauric acid, myristic acid, palmitic acid, stearic acid, oleic acid, arachidic acid, behenic acid, and lignoceric acid. Suitable salts of these free fatty acids suitable for use as gelling agents herein include the potassium, calcium, magnesium, and sodium salts. As noted above, the aqueous-based gel contemplated herein contains no carrageenan as a gelling agent or has an amount of carrageenan as a gelling agent such that the chemical or physical characteristics of the aqueous-based gel are not visually or otherwise measurably different from an aqueous-based gel having the same composition but with no carrageenan as a gelling agent. In an exemplary embodiment, the free fatty acid is present in the aqueous-based gel in an amount in the range of about 1.5 to about 3 weight percent (wt. %) based on the total weight of the aqueous-based gel.

The aqueous-based gel further contains xanthan gum and a gluco- or galactomannan. An example of a suitable glucomannan includes, but is not limited to, konjac gum. Galactomannans suitable for use herein include, but are not limited to, tara gum, guar gum, and locust bean gum. Galactomannans consist of a mannose backbone with galactose side groups and galactomannans with fewer galactose side groups relative to the mannose groups are particularly suitable for use herein. In an exemplary embodiment, the aqueous-based gel contains xanthan gum and locust bean gum, as locust bean gum has a mannose:galactose ratio of 4:1, which is a relatively lower ratio than certain other galactomannans such as guar gum, which has a mannose:galactose ratio of 2:1, although it will be appreciated that guar gum is suitable for use herein.

Further, in an exemplary embodiment, the xanthan gum and gluco- and/or galactomannan are present in the aqueous-based gel in a ratio in the range of about 50:50 to about 75:25, respectively. Xanthan gum, glucomannan and galactomannans are all polysaccharides used as colloids. Thus, it would be expected that simply adding xanthan gum and a glucomannan and/or a galactomannan to a gelling agent would result in a gel having some gel strength but little or no rigid structure and demonstrating unsatisfactory syneresis. It unexpectedly has been found, however, that a ratio of xanthan gum: gluco- and/or galactomannan in the range of about 50:50 to about 75:25 works symbiotically with the C₁₂-C₂₄ free fatty acid. The C₁₂-C₂₄ free fatty acid alone creates a rigid gel that extracts or expels water in the form of syneresis. This syneresis in the C₁₂-C₂₄ free fatty acid is controlled, however, by the synergy of the xanthan gum and the gluco- and/or galactomannan. At ratios of less than 50:50 and greater than 75:25 xanthan gum: gluco- and/or galactomannan, the aqueous-based gel exhibits too low of gel strength for the gel to be free standing, that is, to stand on its own without support of a mold or holder. In addition, at ratios of less than 50:50 and greater than 75:25 xanthan gum:gluco- and/or galactomannan, the aqueous-based gel exhibits an undesirably high syneresis effect. However, the use of a xanthan gum: gluco- and/or galactomannan ratio in the range of about 50:50 to about 75:25 provides an aqueous-based gel capable of free standing with suitable syneresis exhibition.

In another exemplary embodiment, the aqueous-based gel contains a freeze/thaw stabilizer. When a conventional aqueous-based gel is frozen, the water in the gel can freeze and form ice crystals that expand and shear the gel, creating pores. When the conventional aqueous-based gel then thaws, the ice crystals melt and water is released from the gel. This is undesirable as it destroys the aesthetic appearance of the gel. In addition, as with syneresis, if the gel contains dyes or pigments, the dyes and pigments can leave the gel with the water and stain countertops or other support upon which the gel stands. A freeze/thaw stabilizer is a component that causes the aqueous-based gel to reabsorb the water that the gel would otherwise lose to a freeze/thaw cycle. In an exemplary embodiment, the aqueous-based gel contains sodium carboxymethyl cellulose (CMC). Other examples of freeze/thaw stabilizers suitable for use in the aqueous-based gel contemplated herein include hydroxymethyl cellulose and hydroxyethyl cellulose. Iota carrageenan can also be used as a freeze/thaw stabilizer, although the above-described disadvantages of using a carrageenan as a gelling agent apply as well to using a carrageenan as a freeze/thaw stabilizer. The freeze/thaw stabilizer is present in an amount sufficient to cause the aqueous-based gel to absorb water after a freeze/thaw cycle. In an exemplary embodiment, the freeze/thaw stabilizer is present in the aqueous-based gel in an amount in the range of about 0.5 to about 1 wt. % based on the total weight of the aqueous-based gel.

In accordance with an exemplary embodiment, the aqueous-based gel further contains an active ingredient that is delivered to the ambient environment by the aqueous based gel. For example, the active ingredient can be a fragrance such that the aqueous-based gel serves as an air freshener, or the active ingredient can be an insect repellant such as citronella. The active ingredient suitable for use in the aqueous-based gel contemplated herein may comprise one or more naturally derived oils or one or more synthetically derived materials such as aldehydes, ketones, esters, etc. or a combination thereof. Naturally derived active ingredients include, but are not limited to, musk, civet, ambergris, castoreum and the like animal perfumes, abies oil, ajowan oil, almond oil, ambrette seed absolute (“abs.”), angelic root, anise oil, basil oil, bay oil, benzoine resinoid, bergamot oil, birch oil, bois de rose oil, broom abs., cajeput oil, cananga oil, capsicum oil, caraway oil, cardamom oil, carrot seed oil, cassia oil, cedar leaf, cedarwood oil, celery seed oil, cinnamon bark oil, citronella oil, clary sage oil, clove oil, cognac oil, coriander oil, cubeb oil, cumin oil, camphor oil, dill oil, estragon oil, eucalyptus oil, fennel sweet oil, galbanum res., garlic oil, geranium oil, ginger oil, grapefruit oil, hop oil hyacinth abs., jasmin abs., juniper berry oil, labdanum res., lavender oil, laurel leaf oil, lavender oil, lemon oil, lemongrass oil, lime oil, lovage oil, mace oil, mandarin oil, mimosa abs., myrrh abs., mustard oil, narcissus abs., neroli bigarade oil, nutmeg oil, oakmoss abs., olibanum res., onion oil, opoponax res., orange oil, orange flower oil, origanum orris concrete, pepper oil, peppermint oil, peru balsam, petitgrain oil, pine needle oil, rose abs., rose oil, rosemary oil, sandalwood oil, sage oil, spearmint oil, styrax oil, thyme oil, tolu balsam, tonka beans abs., tuberose abs., ylang ylang oil and like vegetable oils, etc. Synthetic active ingredients include but are not limited to pinene, limonene and like hydrocarbons; 3,35-trimethylcyclohexanol, linalool, geraniol, nerol, citronellol, menthol, borneol, borneyl methoxy cyclohexanol, benzyl alcohol, anise alcohol, cinnamyl alcohol, 3-phenyl ethyl alcohol, cis-3-hexenol, terpineol and like alcohols; anethole, musk xylol, isoeugenol, methyl eugenol and like phenols; a-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, citral, citronellal, hydroxycitronellal, benzaldehyde, methyl nonyl acetaldehyde, cinnamic aldehyde, dodecanol, α-hyxylcinnamic aldehyde, undecenal, heliotropin, vanillin, ethyl vanillin and like aldehydes; methyl amyl ketone, methyl β-naphthyl ketone, methyl nonyl ketone, musk ketone, diacetyl, acetyl propionyl, acetyl butyryl, carvone, menthone, camphor, acetophenone, p-methyl acetophenone, ionone, methyl ionone and like ketones; amyl butyrolactone, diphenyl oxide, methyl phenyl glycidate, gamma.-nonyllactone, coumarin, cineole, ethyl methyl phenyl glicydate and like lactones or oxides; methyl formate, isopropyl formate, linalyl formate, ethyl acetate, octyl acetate, methyl acetate, benzyl acetate, cinnamyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, methyl salicylate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, nopyl acetate, β-phenylethyl acetate, trichloromethylphenyl carbonyl acetate, terpinyl acetate, vetiveryl acetate and like esters, and the like. In an embodiment, the active ingredient is present in the aqueous-based gel in an amount in the range of from about 0.7 to about 1.25 wt. % based on the total weight of the aqueous-based gel.

The aqueous-based gel further may include a dye, pigment or other suitable colorant to provide aesthetic appeal to the aqueous-based gel. Such dye may include FD&C and/or D&C Yellow, Red, Blue, Green and Violet, a combination thereof, or any other dye or pigment that is commonly purchased in either powder or liquid than. Dyes and/or pigments are incorporated at levels sufficient to provide light color to deep color to the aqueous-based gel. In an exemplary embodiment, the dye and/or pigment is present in the aqueous-based gel in an amount in the range of about 0.001 to about 0.10 wt. % based on the total weight of the aqueous-based gel.

The aqueous-based gel may also contain a preservative to minimize or prevent dye fading and/or mold or other microbial growth in and/or on the gel. Exemplary microbial preservatives include those containing methylisothiazolinone or other isothiazolinones, such as the Neolone® and Kathon® products from Dow Chemical Company of Midland, Mich. These materials are incorporated at the manufacturers' recommended levels in the air freshener gel to discourage bacterial and mold growth. In an exemplary embodiment, a preservative is present in the aqueous-based gel in an amount in the range of about 0.005 to about 0.015 wt. % based on the total weight of the aqueous-based gel. An ultraviolet inhibitor and/or an antioxidant such as BHT may also be added to the aqueous-based gel to reduce dye fading that may become an issue when the gel is exposed to light. It will be appreciated that the aqueous-based gel can include any additional ingredients that enhance the chemical or physical properties of the aqueous-based gel or facilitate the functions of the above-described ingredients.

The aqueous-based gel further includes water that serves as the carrier and solvent in the aqueous-based gel, in accordance with an embodiment. The water may be in the form of deionized water, tap water, softened water, or the like. Given the amounts of the C₁₂-C₂₄ free fatty acid or a salt thereof, xanthan gum, the gluco- or galactomannan, the freeze/thaw stabilizer, the active ingredient, and any additional ingredient, such as preservative, the remaining ingredient is mostly water. In an exemplary embodiment, water is present in the aqueous-based gel in an amount in the range of from about 94 to about 98 wt. % based on the total weight of the aqueous-based gel.

In another exemplary embodiment, a method for manufacturing an aqueous-based gel capable of free standing includes mixing a C₁₂-C₂₄ fatty acid or a salt thereof, a freeze thaw stabilizer, xanthan gum, and a gluco- or galactomannan with water at a temperature sufficient to form a homogeneous mixture. The xanthan gum and the gluco- or galactomannan are present in a xanthan gum:gluco- or galactomannan ratio in a range of about 50:50 to about 75:25. Prior to addition of the solid ingredients, the water is heated and maintained at a temperature at or above the gelling point of the mixture. This gelling point temperature can be determined by empirical evidence. For example, in one embodiment using sodium stearate, CMC, xanthan gum and locust bean gum, the water is heated to and maintained at 80° C. The C₁₂-C₂₄ fatty acid or a salt thereof, the freeze/thaw stabilizer, the xanthan gum, and the gluco- or galactomannan can be added individually to the water in any order or the solid ingredients can be combined together first to form a preformed powder mixture and this mixture can be added to the water. Any of the C₁₂-C₂₄ fatty acids or salts thereof, the freeze/thaw stabilizers and the gluco- or galactomannans described above can be utilized in the method described herein. Any preservative, dyes and other non-highly volatile ingredients can also be added at this time. The ingredients are mixed for a time and at a mixing speed suitable to obtain a homogeneous liquid mixture. For example, the ingredients are mixed for about 5 to about 30 minutes, for example, about 8 to about 15 minutes. The ingredients can be mixed using any suitable high shear mixing method that is suitable for hydrating the colloids, for example, using a high shear mixer, a high shear emulsifier, and the like. During high shear mixing, air can be introduced into the liquid mixture, the amount of air introduced dependent on the speed of the high shear mixing. It has been unexpectedly discovered that the aqueous-based gel contemplated herein is able to retain more air than a comparable carrageenan-based gel, resulting in a lower density product. Thus, the mass of the aqueous-based gel contemplated herein required to fill a volume, such as a mold, is less than the mass of a comparable carrageenan-based gel required to fill the same volume. This can result in a reduction in production costs relative to prior art carrageenan systems. In this regard, in an embodiment, the amount of fragrance may be increased relative to the total weight of the aqueous-based gel to maintain the fragrance effect relative to a comparable carrageenan-based gel.

If a fragrance or other highly volatile active ingredient is to be used in the aqueous-based gel contemplated herein, the method continues with the addition of the fragrance or other active ingredient and a second amount of water to the homogeneous liquid mixture. In accordance with an embodiment, the second amount of water is at a temperature equal to or greater than the temperature of the homogeneous liquid mixture, for example, 80° C., to maintain the mixture in a liquid state. Mixing is continued for an amount of time sufficient to thoroughly integrate the fragrance or additive into the liquid so as to obtain a homogeneous mixture but not so long as to cause the fragrance or additive to vaporize or “flash-off.” The liquid mixture can be mixed using any of the methods described above for mixing. In one embodiment, the mixture is mixed by high shear mixing for 1 to 5 minutes, for example, 3 minutes.

Next, the homogeneous liquid mixture is cooled actively or passively. In one embodiment, the homogeneous liquid mixture is poured into one or more molds or other containers. In another embodiment, the homogeneous liquid mixture then is cooled passively by subjecting the homogeneous liquid mixture to ambient air at room temperature, that is, at temperatures in the range of about 18° C. to about 30° C., for a time sufficient for the homogeneous liquid mixture to form a solid gel. In an exemplary embodiment, the homogeneous liquid mixture is subjected to room temperature for no less than two hours. In another exemplary embodiment, the homogeneous liquid mixture is cooled actively by subjecting the homogeneous liquid mixture to temperatures below 18° C. for a time sufficient for the homogeneous liquid mixture to form a solid gel. The homogeneous liquid mixture can be actively cooled by methods such as insertion in a refrigeration unit and/or exposure to moving cooled air via fans. Once the homogeneous liquid mixture has formed a solid gel, it can be removed from the cooling method or the cooling method can be terminated. Next, the aqueous-based gel can be removed from the mold or container to form free standing gel as may be desired.

### EXAMPLES

The following are examples of aqueous-based gels capable of free standing as contemplated herein with each of the components set forth in weight percent, unless otherwise noted. The examples are provided for illustration purposes only and are not meant to limit in any way the various embodiments of the aqueous-based gels, free-standing air fresheners and methods for fabricating aqueous-based gels, as contemplated herein.

The following aqueous-based gels were produced by combining sodium stearate, xanthan gum and locust bean gum in a 50:50 ratio of xanthan gum:locust bean gum, and CMC in accordance with the following weight percents. Particularly, dry blends of the sodium stearate, xanthan gum and locust bean were prepared according to the weight percents below. For each of the aqueous-based gels, a dry blend and the Neolone M10 were then added to a vessel containing water held at 80° C. while the mixture was stirred using an Admix® OPLB-300 mixer at a speed of 3000 to 5000 revolutions per minute (rpm). Neolone M10 is a preservative containing 2-methyl-4-isothiazolin-3-one and 2-methyl-3(2H)isothiazolinone available from Dow Chemical Company of Midland, Michigan. The mixtures were subjected to high shear mixing for about 8 to about 13 minutes. After this time, water at 80° C. and the fragrance were added to the mixture and high shear mixing continued for an additional 3 minutes. The mixture was then poured into molds and allowed to cool for 24 hours. It was found that, while 198g of a conventional carrageenan formula using Kappa and Iota carrageenan was required to fill a mold to a particular fill point, only 170 g of Embodiment 4 was required to fill the same mold to the same fill point. As noted above, without being bound by theory, it is believed that the sodium stearate in Embodiment 4 allowed for a higher amount of air to be incorporated into the mixture during high shear mixing than carrageen allowed for in the carrageen-based formula. The weight percentage of the fragrance of Embodiment 4 was increased relative to the total weight of the aqueous-based gel to maintain the fragrance effect of the aqueous-based gel.

| | | Embodiment | Embodiment | Embodiment |
|---|---|---|---|---|
| Material | Embodiment 1 | 2 | 3 | 4 |
| Water | 95.1400 | 95.3200 | 95.2600 | 95.1800 |
| Na Stearate | 2.5000 | 2.5000 | 2.5000 | 2.5000 |
| CMC | 0.7500 | 0.7500 | 0.7500 | 0.7500 |
| Xanthan | 0.3000 | 0.3000 | 0.3000 | 0.3000 |
| Gum | | | | |
| Locust Bean | 0.3000 | 0.3000 | 0.3000 | 0.3000 |
| Gum | | | | |
| Fragrance | 1.0000 | 0.8200 | 0.8800 | 0.9600 |
| Neolone | 0.0100 | 0.0100 | 0.0100 | 0.0100 |
| M10 | | | | |
| Total | 100.0000 | 100.0000 | 100.00 | 100.0000 |

Accordingly, aqueous-based gels capable of free standing, free-standing air fresheners, and methods for manufacturing aqueous-based gels have been provided herein. While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. An aqueous-based gel capable of free standing, the aqueous-based gel comprising:
a C₁₂-C₂₄ fatty acid or a salt thereof;
a freeze/thaw stabilizer;
xanthan gum;
a gluco- and/or galactomannan; and
water,
wherein the xanthan gum and the gluco- and/or galactomannan are present in the aqueous-based gel in a xanthan gum:gluco- and/or galactomannan ratio in a range of about 50:50 to about 75:25 and wherein the aqueous-based gel is carrageenan-free.

2. The aqueous-based gel of claim 1, wherein the C₁₂-C₂₄ fatty acid or a salt thereof comprises sodium stearate.

3. The aqueous-based gel of claim 1, wherein the C₁₂-C₂₄ fatty acid or a salt thereof is present in the aqueous-based gel in an amount in a range of about 1.5 to about 3 weight percent based on a total weight of the aqueous-based gel.

4. The aqueous-based gel of claim 1, wherein the freeze/thaw stabilizer comprises sodium carboxymethyl cellulose.

5. The aqueous-based gel of claim 1, wherein the freeze/thaw stabilizer is present in the aqueous-based gel in an amount in a range of about 0.5 to about 1 wt. % based on a total weight of the aqueous-based gel.

6. The aqueous-based gel of claim 1, wherein the gluco- and/or galactomannan is locust bean gum.

7. The aqueous-based gel of claim 1, wherein the water is present in an amount in a range of about 94 to about 98 weight percent based on a total weight of the aqueous-based gel.

8. A free-standing air freshener comprising:
a C₁₂-C₂₄ fatty acid or a salt thereof;
sodium carboxymethyl cellulose;
xanthan gum;
a gluco- and/or galactomannan;
an active ingredient; and
water,
wherein the xanthan gum and the gluco- and/or galactomannan are present in the free-standing air freshener in a xanthan gum:gluco- and/or galactomannan ratio in a range of about 50:50 to about 75:25.

9. The free-standing air freshener of claim 8, wherein the C₁₂-C₂₄ fatty acid or a salt thereof comprises sodium stearate.

10. The free-standing air freshener of claim 8, wherein the C₁₂-C₂₄ fatty acid or a salt thereof is present in the free-standing air freshener in an amount in a range of about 1.5 to about 3 weight percent based on a total weight of the free-standing air freshener.

11. The free-standing air freshener of claim 8, wherein the sodium carboxymethyl cellulose is present in the free-standing air freshener in an amount in a range of about 0.5 to about 1 weight percent based on a total weight of the free-standing air freshener.

12. The free-standing air freshener of claim 8, wherein the gluco- and/or galactomannan is locust bean gum.

13. The free-standing air freshener of claim 8, wherein the water is present in an amount in a range of about 94 to about 98 weight percent based on a total weight of the free-standing air freshener.

14. A method for manufacturing an aqueous-based gel capable of free standing, the method comprising the steps of:
mixing a C₁₂-C₂₄ fatty acid or a salt thereof, a freeze/thaw stabilizer, xanthan gum, and a gluco- or galactomannan with water at a temperature sufficient to form a homogeneous liquid mixture, wherein the xanthan gum and the gluco- and/or galactomannan are present in a xanthan gum:gluco- and/or galactomannan ratio in a range of about 50:50 to about 75:25; and
cooling the homogeneous liquid mixture actively or passively wherein the aqueous-based gel is carrageenan free.

15. The method of claim 14, wherein mixing the C₁₂-C₂₄ fatty acid or a salt thereof comprises mixing sodium stearate.

16. The method of claim 14, wherein the C₁₂-C₂₄ fatty acid or a salt thereof is mixed in an amount in a range of about 1.5 to about 3 weight percent based on a total weight of the aqueous-based gel.

17. The method of claim 14, wherein mixing the freeze/thaw stabilizer comprises mixing sodium carboxymethyl cellulose.

18. The method of claim 14, wherein the freeze/thaw stabilizer is mixed in an amount in a range of about 0.5 to about 1 weight percent based on a total weight of the aqueous-based gel.

19. The method of claim 14, wherein mixing the gluco- or galactomannan comprises mixing locust bean gum.

20. The method of claim 14, wherein the mixing comprises high shear mixing such that air is introduced into the homogeneous liquid mixture.
